(19) United States
(12) Patent Application Publication
(10) Pub. No.: US 2026/0207575 A1
(43) Pub. Date: Jul. 23, 2026

(54) PRIDOPIDINE FOR THE TREATMENT OF JUVENILE HUNTINGTON DISEASE

(71) Applicant: Prilenia Neurotherapeutics Ltd., Yakum (IL)

(72) Inventors: Michael HAYDEN, Yakum (IL); Michal GEVA, Even-Yehuda (IL); Yigal Paul GOLDBERG, Vancouver (CA)

(73) Assignee: Prilenia Neurotherapeutics Ltd., Yakum (IL)

(21) Appl. No.: 19/142,208

(22) PCT Filed: Dec. 27, 2023
(86) PCT No.: PCT/IL2023/051324

§ 371 Date: Jun. 22, 2025

Related U.S. Application Data
(60) Provisional application No. 63/477,227, filed on Dec. 27, 2022.

(51) Int. Cl. A61K 31/451; A61K 31/4745; A61P 25/28

(52) U.S. Cl. CPC A61K 31/451; A61K 31/4745; A61P 25/28

(57) ABSTRACT

Provided herein a method of treating Juvenile Huntington disease in a subject in need thereof comprising orally administering a pharmaceutical composition comprising pridopidine and/or its analog or a pharmaceutically acceptable salt thereof.

### FIELD OF THE INVENTION

Provided herein a method of treating a Juvenile Huntington disease comprising orally administering a pharmaceutical composition comprising pridopidine and/or its analog or a pharmaceutically acceptable salt thereof.

### BACKGROUND OF INVENTION

### Huntington Disease

Huntington disease (HD) is a fatal neurodegenerative disorder with an autosomal dominant mode of inheritance. Symptoms typically begin between the ages of 40 and 60, and the disease progresses over 10 to 30 years eventually leading to death. The prevalence of HD in the general population is approximately 10 per 100,000, about 100,000 people worldwide. Approximately 5% of HD subjects develop clinical features prior to the age of 20, and these are classified as juvenile-onset HD (JHD). The prevalence of JHD is approximately 0.5 per 100,000.

Adult-onset and JHD share the same causative mutation, an expanded tract of at least 36 CAG repeats in the HTT gene. However, the number of repeats is highly variable, and correlates with disease severity and progression (higher repeat length correlates with an earlier onset). JHD is associated with a high number of CAG repeats, usually over fifty. Additionally, there are significant phenotypic differences between the adult's onset HD and JHD.

HD is associated with a triad of motor, behavioral, and cognitive symptoms. Motor disturbances are a defining feature of the disease. Disability and disease severity best correlate with negative functional and motor features such as decline in functional capacity and impairment in fine motor skills and gross motor coordination skills, including speech difficulties, gait, and postural dysfunction (Mahant et al., Huntington's disease: clinical correlates of disability and progression. Neurology. 2003 Oct. 28; 61(8):1085-92. doi: 10.1212/01.wnl.0000086373.32347.16. PMID: 14581669).

Compared to adult-onset HD, which is primarily associated with chorea as the defining motor feature, JHD manifests with motor symptoms including rigidity, dystonia and bradykinesia, as well as behavioral problems and developmental delay. JHD patients suffer from more severe symptoms, and a faster disease progression (Quarrell et al., Managing juvenile Huntington's disease. Neurodegener Dis Manag. 2013 Jun. 1; 3(3):10.2217/nmt.13.18).

Neuropathologically, JHD is primarily characterized by neuronal loss in the striatum and cortex. However, many other nuclei including the globus pallidus, thalamus, hypothalamus, subthalamic nucleus, substantia nigra and cerebellum are also affected. In addition, diffusion tensor imaging has demonstrated pathology of the white matter in pre- and early symptomatic patients (Raymond et al., Pathophysiology of Huntington's disease: time-dependent alterations in synaptic and receptor function. Neuroscience. 2011 Dec. 15; 198:252-73). The pathology in JHD is typically more extensive and more severe than in classical adult-onset HD (Quarrell et al, 2013).

A number of medications are prescribed to help manage the motor, cognitive and behavioral problems associated with HD. However, these are all directed at specific symptoms. No registered drugs are available that favorably modulate the multifaceted symptoms of HD, resulting in inexorable motor, cognitive and behavioral decline throughout the course of the disease.

The different presentation of JHD means that different treatments are likely to be used in the management of JHD versus adult-onset HD (Robertson, Lisa, et al. “Current pharmacological management in juvenile Huntington's disease.” PLoS currents 4 (2012)). For example, the drugs tetrabenazine (TBZ) and deutetrabenazine (dTBZ) are approved for the treatment of chorea in adult-onset HD. However, TBZ and dTBZ are not used in JHD, as its safety and efficacy have not been demonstrated in children.

Altogether, there is no effective treatment for JHD and patients have progressive, uncontrolled and inexorable evolution of the disease with deterioration of the neurologic status, loss of ability for self-caring in daily activities and untimely death.

As all current therapies are only symptomatic, a therapy that prevents or delays the development of disability would represent a substantial improvement over the currently available therapies. As such, there is a significant unmet medical need to develop medications to treat JHD.

### Pridopidine

Pridopidine (4-[3-(methylsulfonyl)phenyl]-1-propyl-piperidine) (formerly known as ACR16) is a drug in clinical development for the treatment of HD. Pridopidine has a selective and high affinity for the sigma-1 receptor (S1R, binding IC50~100 nM), with low-affinity binding to other receptors, including the dopamine D2/D3 receptors (in the micromolar range).

The Sigma-1 receptor (S1R) is an endoplasmic reticulum (ER) chaperone protein implicated in calcium homeostasis, cellular bioenergetics, neuroplasticity and neuroprotection.

### SUMMARY OF THE INVENTION

In some embodiments, provided herein a method of treating or lessening the progression of Juvenile Huntington Disease (JHD) in a subject, comprising orally administering to the subject a pharmaceutical composition comprising pridopidine, its analog or a pharmaceutically acceptable salt thereof.

In some embodiments, the juvenile Huntington Disease subject has a mutation in the Huntingtin (Htt) gene of 50 or more CAG repeats. In some embodiments the JHD subject has 55 or more CAG repeats.

In some embodiments, the JHD subject experience symptom onset before the age of 16. In some embodiments, symptom onset was before age 18. In another embodiment, symptom onset was before age 20. In another embodiment, symptom onset was before age 21.

In some embodiments, the JHD subject is a premanifest or prodromal JHD subject.

The recently created HD Integrated Staging System (ISS) defines specific stages based on distinct clinical landmarks and differentiates between presymptomatic, prodromal and manifest HD patients.

### HD Integrated Scoring System (HD-ISS)

In some embodiments, a prodromal JHD subject is characterized by an ISS stage of 1 or 2. In some embodiments, a pre-symptomatic JHD subject has an ISS score of 0.

The HD-ISS is an evidence-based staging system that addresses all stages of HD. The ISS defines landmark assessments and cut-off values to identify critical transitions in disease stages. HD-ISS has 4 distinct stages. ISS Stage 0 (pre-symptomatic) includes all HD gene carriers for whom there is no detectable change in pathological markers, signs or symptoms related to HD. In ISS stage 1, pathological changes indicating neurodegeneration and an onset of specific signs can be detected, specifically changes to the volume of the caudate and putamen brain substructures. ISS Stage 2 is defined by the presence of definite clinical signs and symptoms, both cognitive and motor without any decline in function. ISS Stage 3 (manifest) is accompanied by a decline in functional capacity or independent scale.

In some embodiments, provided herein a method of maintaining or reducing one or more symptoms of Juvenile Huntington Disease (JHD) in a subject, comprising orally administering to the subject a pharmaceutical composition comprising pridopidine and/or its analog or a pharmaceutically acceptable salt thereof wherein the one or more symptoms are selected from the group consisting of depression, anxiety, apathy, impulsivity, aggression, motor function impairment, cognitive impairment, a physical symptom, seizures, a mental symptom, an emotional symptom, a behavioral symptom, and reduced lifespan.

In some embodiments, provided herein a method of maintaining, improving, or lessening the decline of motor, cognitive or behavioral function in a Juvenile Huntington disease subject, comprising orally administering to the subject a pharmaceutical composition comprising pridopidine and/or its analog or a pharmaceutically acceptable salt thereof.

### DETAILED DESCRIPTION OF THE INVENTION

This invention provides a method of treating or lessening the progression of Juvenile Huntington disease (JHD) in a subject, comprising administering to the subject a pharmaceutical composition comprising pridopidine and/or its analog or a pharmaceutically acceptable salt thereof.

Huntington disease (HD) is caused by an expansion of a cytosine-adenine-guanine (CAG) repeat within the coding region of the Huntington (Htt) gene. The gene encodes a large protein (~350 kDa) called huntingtin (HTT) that is highly conserved and expressed ubiquitously throughout the body. The wild-type gene codes for a polyglutamine stretch near its amino terminus in the range 6 to 35 repeats. HD is associated with 36 or more CAG repeats. The mutation results in an elongated stretch of polyglutamines in the first exon of the huntingtin protein. Definite clinical manifestation will occur when the number of repeats exceeds 39, while the range 36-39 leads to incomplete penetrance of the disease or to a late onset. An inverse correlation has been well characterized between the length of the CAG repeat and the age at onset, determined by the first motor manifestation. The longer the CAG repeat, the earlier the onset (Andrew et al. The relationship between trinucleotide (CAG) repeat length and clinical features of Huntington's disease. Nat Genet. 1993 August; 4(4):398-403). In juvenile HD (JHD), the disease onset is at the age of ≤20 years, and the repeat often exceeds 50 CAGs (Roos R A. Huntington's disease: a clinical review. Orphanet J Rare Dis. 2010 Dec. 20; 5:40; Raymond et al., Pathophysiology of Huntington's disease: time-dependent alterations in synaptic and receptor function. Neuroscience. 2011 Dec. 15; 198:252-73; Quarrell et al., The Prevalence of Juvenile Huntington's Disease: A Review of the Literature and Meta-Analysis. PLoS Curr. 2012 Jul. 20; 4:e4f8606b742ef3; Telenius et al. Molecular analysis of juvenile Huntington disease: the major influence on (CAG)n repeat length is the sex of the affected parent. Hum Mol Genet. 1993 October; 2(10):1535-40).

Neuropathologically, JHD is primarily characterized by neuronal loss in the striatum and cortex. However, many other nuclei including the globus pallidus, thalamus, hypothalamus, subthalamic nucleus, substantia nigra and cerebellum are also affected. In addition, diffusion tensor imaging has demonstrated pathology of the white matter in pre- and early symptomatic patients (Raymond et al, 2011). The pathology in JHD is typically more extensive and more severe than in classical adult-onset HD (Quarrell et al, 2013).

The prevalence of JHD (onset at age≤20) is considerably lower than that of adult-onset HD. JHD is exceptionally rare and accounts for approximately 5% of all HD cases. This yields a conservatively estimated prevalence of JHD of 0.5 per 100,000 people when assuming an overall HD prevalence of 10 per 100,000.

### JHD

JHD is defined by clinical onset≤20 years. Childhood-onset JHD begins in the first decade of life (≤10 years of age), while adolescent-onset starts in the second decade (11-20 years of age) of life (Quarrell et al, 2012; Quarrell et al, 2013). The diagnosis of JHD is often substantially delayed due to the relative rarity of JHD, lack of physician awareness of JHD, the complex and diverse symptom presentation and initial non-specificity of presentation (Achenbach et al., Clinical Manifestation of Juvenile and Pediatric HD Patients: A Retrospective Case Series. Brain Sci. 2020 Jun. 3; 10(6):340; Ribal et al., Psychiatric and cognitive difficulties as indicators of juvenile huntington disease onset in 29 patients. Arch Neurol. 2007 June; 64(6):813-9). One study identified a mean delay in diagnosis of 9 years (Ribai et al, 2007). Furthermore, JHD usually has a faster progression and shorter duration than adult-onset HD (Foroud et al., Differences in duration of Huntington's disease based on age at onset. J Neurol Neurosurg Psychiatry. 1999 January; 66(1):52-6, Quarrell et al., 2013, Foroud T, Gray J, Ivashina J, Conneally P M. Differences in duration of Huntington's disease based on age at onset. J Neurol Neurosurg Psychiatry. 1999 January; 66(1):52-6; Latimer et al., Neuropathological Comparison of Adult Onset and Juvenile Huntington's Disease with Cerebellar Atrophy: A Report of a Father and Son. J Huntingtons Dis. 2017; 6(4):337-348).

### Childhood-Onset JHD

The clinical features of JHD when presenting in first decade of life (childhood-onset JHD) are very different to adult-onset HD with a more severe and rapidly progressing phenotype. The clinical picture is often one of cognitive impairment (especially for executive function) with declining school performance, and behavioral abnormalities. This is further exacerbated by speech and language problems.

In contrast to adult-onset HD where the initial motor manifestations are hyperkinetic with chorea, childhood-onset JHD patients have a different phenotype with severe bradykinesia, rigidity and dystonia as well as ataxia as their initial motor manifestation. Presentation with rigidity is a distinguishing feature of JHD not seen in adult-onset HD (Ribai et al, 2007; Quigley J. Juvenile Huntington's Disease: Diagnostic and Treatment Considerations for the Psychiatrist. Curr Psychiatry Rep. 2017 February; 19(2):9).

In addition, epilepsy is common and in some patients may initially lead to confusion with progressive myoclonic epilepsies (Thakor et al., Juvenile Huntington's disease masquerading as progressive myoclonus epilepsy. Epilepsy Behav Rep. 2021 Jul. 15; 16:100470.) Epileptic seizures are present in approximately one third of JHD cases and are much more likely to occur in childhood-onset JHD than adolescent-onset (Cloud et al., Seizures in juvenile Huntington's disease: frequency and characterization in a multicenter cohort. Mov Disord. 2012 December; 27(14):1797-800). There is a corresponding increased seizure risk, the younger the age of disease onset. Seizures are often generalized tonic-clonic (Cloud et al, 2012). In the initial stage of the disease, seizures in childhood-onset JHD are typically well controlled. However, as the disease progresses, they become drug-resistant (Khair et al., Drug-Resistant Epilepsy in Children with Juvenile Huntington's Disease: A Challenging Case and Brief Review. Qatar Med J. 2020 Jul. 14; 2020(1):18. Gonzalez-Alegre P, Afifi A K. Clinical characteristics of childhood-onset (juvenile) Huntington disease: report of 12 patients and review of the literature. J Child Neurol. 2006 March; 21(3):223-9). Initial presentation with seizures is a further distinguishing feature of JHD not seen in adult-onset HD.

The burden of psychiatric symptoms in JHD is considerable and includes depression, anxiety, impulsivity, and aggression (Quigley 2017). Behavioral disturbances are a hallmark and often the cardinal manifestation of childhood-onset JHD. These can be very debilitating for the child and family impacting relationships and the ability to function socially and emotionally. Patient can be initially misdiagnosed as having attention-deficit hyperactivity disorder (ADHD) (Gonzalez-Alegre and Afifi, 2006).

### Adolescent-Onset JHD

The clinical features of individuals who onset in the second decade (adolescent-onset JHD) show some phenotypic differences compared to childhood-onset features. For example, while chorea is seldom seen when JHD presents initially in the first decade, it may be seen in patients who onset in the second decade (adolescent-onset JHD) (Roos 2010; Quarrell et al., 2013; Oosterloo et al., Diagnosing Juvenile Huntington's Disease: An Explorative Study among Caregivers of Affected Children. Brain Sci. 2020 Mar. 7; 10(3):155.) Seizures can also be seen in adolescent-onset HD (Cloud et al, 2012).

In adolescent-onset JHD, cognitive decline is commonly present with deteriorating academic performance at school. There are a wide range of behavioral and psychiatric manifestations including drug and alcohol abuse, depression with suicide attempts, hyperactivity, or personality disorders (Nance M A. Clinical aspects of CAG repeat diseases. Brain Pathol. 1997 July; 7(3):881-900; Ribai et al., 2007). As the disease progresses, it evolves into a phenotype similar to the childhood-onset patients (Hansotia et al., Juvenile Huntington's chorea. Neurology. 1968 March; 18(3):217-24; van Dijk J G et al., Juvenile Huntington disease. Hum Genet. 1986 July; 73(3):235-9). Significant oropharyngeal dysfunction is often seen, while other features in adolescent-onset JHD may include rigidity, dystonia, and chorea.

This invention provides a method of maintaining or reducing one or more symptoms of Juvenile Huntington disease in a subject, comprising administering to the subject a pharmaceutical composition comprising pridopidine and/or its analog or a pharmaceutically acceptable salt thereof. In an embodiment, the one or more symptoms are selected from the group consisting of depression, anxiety, motor function impairment, cognitive impairment, a physical symptom, a mental symptom, an emotional symptom, a behavioral symptom, impairment of the patient's functional capacity and reduced lifespan.

In one embodiment, provided herein is a method of maintaining, improving, or lessening the decline of motor function in a human subject afflicted with Juvenile Huntington disease, said method comprises orally administering to the patient a pharmaceutical composition comprising pridopidine and/or its analog or a pharmaceutically acceptable salt thereof.

In some embodiments, the method comprises maintaining, improving, or lessening the decline of motor function in said subject.

In some embodiments, the method comprises reducing or lessening seizures in a Juvenile Huntington Disease subject.

In one embodiment, provided herein a method of improving, preventing the worsening or reducing depression in a Juvenile Huntington Disease subject. In another embodiment, provided herein a method of improving, preventing the worsening or reducing anxiety in a Juvenile Huntington Disease subject. In another embodiment, provided herein a method of maintaining or reducing impulsivity in a Juvenile Huntington Disease subject. In another embodiment, provided herein a method of improving, preventing the worsening or reducing aggression in a Juvenile Huntington Disease subject.

In one embodiment, provided herein a method of maintaining or improving or lessening the decline of motor function impairment in a Juvenile Huntington Disease subject. In one embodiment, provided herein a method a maintaining or improving or lessening the decline of cognitive impairment in a Juvenile Huntington Disease subject. In one embodiment, provided herein a method a maintaining or improving or lessening the decline of memory impairment in a Juvenile Huntington Disease subject. In one embodiment, provided herein a method a maintaining or improving or lessening the decline of learning ability in a Juvenile Huntington Disease subject.

In one embodiment, provided herein a method a maintaining or improving or lessening the decline of a physical symptom in a Juvenile Huntington Disease subject. In one embodiment, provided herein a method a maintaining or improving or lessening the decline of a mental symptom in a Juvenile Huntington Disease subject. In one embodiment, provided herein a method of improving, preventing the worsening or reducing of an emotional symptom in a Juvenile Huntington Disease subject. In one embodiment, provided herein a method a maintaining or reducing of behavioral symptom in a Juvenile Huntington Disease subject. In one embodiment, provided herein a method a maintaining improving or lessening the decline of reduced lifespan in a Juvenile Huntington Disease subject.

Further provided is a method of improving, maintaining, or slowing the decline of, a human patient's cognitive domains comprising orally administering to the patient a pharmaceutical composition comprising pridopidine and/or its analog or a pharmaceutically acceptable salt thereof, so as to thereby improve, maintain, or slow the decline of a human patient's cognitive domains. In some embodiments, the patient is a juvenile Huntington disease (JHD) patient. A patient's cognitive domains may also be the patient's cognitive performance across a variety of domains.

The human patient's cognitive domains may be measured, for example, by the cognitive assessment battery (CAB) and/or the Hopkins Verbal Learning Test-Revised (HVLT-R). The cognitive domains may also be measured by the trail making test B (TMT-B). The cognitive domains may also be measured by the HD Cognitive Assessment Battery (HD-CAB), which includes 6 tests. The cognitive measures may also be measured by the Leiter International Performance Scale and the Peabody Picture Vocabulary Test.

Further provided is a method of improving or maintaining motor ability in a human patient comprising orally administering to the patient a pharmaceutical composition comprising pridopidine and/or its analog or a pharmaceutically acceptable salt thereof, so as to thereby improve motor ability in the human patient. In some embodiments, an improvement in motor ability constitutes less bradykinesia, improved ataxia and/or gait, less rigidity, less dystonia, and/or better coordination, eye movements, hand movements. In some embodiments, the patient is a juvenile Huntington disease (HD) patient.

The invention further provides method of improving or maintaining a human patient's mobility comprising orally administering to the patient a pharmaceutical composition comprising pridopidine and/or its analog or a pharmaceutically acceptable salt thereof, so as to thereby improve or maintain the human patient's mobility. In some embodiments, the patient is a Juvenile Huntington disease (HD) patient.

The invention also provides a method of reducing or maintaining a human patient's involuntary movements comprising orally administering to the patient a pharmaceutical composition comprising pridopidine and/or its analog or a pharmaceutically acceptable salt thereof, so as to thereby reduce or maintain a human patient's involuntary movements. In some embodiments, the patient is a Juvenile Huntington disease (JHD) patient.

The motor ability may be measured, for example, by the UHDRS Total Motor Score (TMS) score, the UHDRS TMS score minus chorea or UHDRS TMS score minus dystonia.

The invention also provides a method of reducing or maintaining the level of dystonia in a human patient comprising periodically orally administering to the patient a pharmaceutical composition comprising pridopidine and/or its analog or a pharmaceutically acceptable salt thereof, so as to thereby reduce or maintain the level of dystonia in a human patient. In some embodiments, the patient is a Juvenile Huntington disease (HD) patient.

The worsening of dystonia may also be slowed. Accordingly, the invention provides a method of reducing, maintaining, or slowing the worsening of, dystonia in a human patient comprising orally administering to the patient a pharmaceutical composition comprising pridopidine and/or its analog or a pharmaceutically acceptable salt thereof, so as to thereby reduce, maintain, or slow the increase of, dystonia in a human patient. In some embodiments, the patient is a Juvenile Huntington disease (HD) patient.

The human patient's dystonia may be measured by the UHDRS TMS dystonia score.

The invention also provides a method of reducing or maintaining gait and balance in a human patient comprising periodically orally administering to the patient a pharmaceutical composition comprising pridopidine and/or its analog or a pharmaceutically acceptable salt thereof, so as to thereby reduce or maintain gait and balance in a human patient. In some embodiments, the patient is a Juvenile Huntington disease (HD) patient.

The decline in gait and balance may also be slowed. Accordingly, the invention provides a method of reducing, maintaining, or slowing the decline of gait and balance in a human patient comprising orally administering to the patient a pharmaceutical composition comprising pridopidine and/or its analog or a pharmaceutically acceptable salt thereof, so as to thereby reduce, maintain, or slow the decline of gait and balance in a human patient. In some embodiments, the patient is a Juvenile Huntington disease (HD) patient.

The human patient's gait and balance may be measured by the UHDRS TMS gait and balance score.

The invention also provides a method of reducing or maintaining the level of chorea in a human patient comprising periodically orally administering to the patient a pharmaceutical composition comprising pridopidine and/or its analog or a pharmaceutically acceptable salt thereof, so as to thereby reduce or maintain the level of chorea in a human patient. In some embodiments, the patient is a Juvenile Huntington disease (HD) patient.

The worsening of chorea may also be slowed. Accordingly, the invention provides a method of reducing, maintaining, or slowing the worsening of chorea in a human patient comprising orally administering to the patient a pharmaceutical composition comprising pridopidine and/or its analog or a pharmaceutically acceptable salt thereof, so as to thereby reduce, maintain, or slow the increase of chorea in a human patient. In some embodiments, the patient is a Juvenile Huntington disease (HD) patient.

The human patient's chorea may be measured by the UHDRS TMS chorea score.

In some embodiments, the patient's motor function is evaluated by digitomotography (finger tapping). In some embodiments, the motor function is evaluated by the Quantitative Motor (Q-Motor) battery of tests.

### Quantitative-Motor (Q-Motor)

Q-Motor is an objective assessment of specific motor functions that utilizes pre-calibrated and temperature-independent force transducers and three-dimensional position sensors to provide standardized, unbiased measurements.

The Q-motor test was recently tested in children, showing that it is sensitive to age-related changes in motor abilities. Finger-tapping speed is directly correlated to age, with young children tapping slower and with greater irregularity than older children (Van Der Plas et al. 2019). Thus, it may be used to detect developmental delays.

In another embodiment a JHD subject has decreased Q-Motor finger tap speed frequency, decreased Q-Motor finger tap Inter-Onset-Interval, decreased Q-Motor finger tap Inter-Tap-Interval, decreased Q-Motor inter-peak interval, decreased Q-Motor pronate/supinate hand tapping frequency, decreased Q-Motor hand tapping inter-onset interval, decreased grip force, decreased tongue force or any combination thereof compared to healthy controls.

Further provided is a method of improving or maintaining motor ability as assessed by Q-Motor in a human patient comprising orally administering to the patient a pharmaceutical composition comprising pridopidine and/or its analog or a pharmaceutically acceptable salt thereof, so as to thereby improve Q-Motor ability in the human patient. In some embodiments, an improvement in motor ability constitutes improved Q-Motor finger tap Inter-Tap Interval, increased or maintained Q-motor inter-peak interval, improved or maintained Q-Motor pronate/supinate hand tapping frequency, improved or maintained Q-Motor hand-tapping inter-onset interval, improved or maintained grip force, improved or maintained tongue force or any combination thereof. In some embodiments, the patient is a juvenile Huntington disease (HD) patient.

The invention further provides a method of improving or maintaining a human patient's behavior and/or psychiatric state comprising orally administering to the patient a pharmaceutical composition comprising pridopidine and/or its analog or a pharmaceutically acceptable salt thereof, so as to thereby improve or maintain the human patient's behavior and/or psychiatric state. In some embodiments, the patient is a Juvenile Huntington disease (JHD) patient.

The human patient's behavior and/or psychiatric state may be measured, for example, by the Problem Behaviors Assessment (PBA) total score. The human patient's behavior and/or psychiatric state may also be measured by the Problem Behaviors Assessment for depressed mood. The human patient's behavior and/or psychiatric state may also be measured by the Problem Behaviors Assessment for irritability. The human patient's behavior and/or psychiatric state may also be measured by the Problem Behaviors Assessment for lack of initiative or apathy. The human patient's behavior and/or psychiatric state may be measured, for example, by the Problem Behaviors Assessment for obsessive-compulsiveness. The human patient's behavior and/or psychiatric state may also be measured by the Problem Behaviors Assessment for disoriented behavior.

Further provided is a method of improving or lessening decline of lack of initiative or apathy in a human HD patient comprising orally administering to the patient a pharmaceutical composition comprising pridopidine and/or its analog or a pharmaceutically acceptable salt thereof, so as to thereby improve or lessen decline of lack of initiative or apathy in the patient. In some embodiments, the patient is a Juvenile Huntington disease (JHD) patient.

In some embodiments, the invention further provides a pharmaceutical composition comprising pridopidine and/or its analog or a pharmaceutically acceptable salt thereof for use in (1) (a) delaying onset or slowing the clinical progression of HD in a JHD patient, (b) reducing dystonia or maintaining a level of dystonia in a JHD patient, (c) treating limb dystonia in a in a JHD patient, (d) preventing the slowing, the reduction in amplitude, or the impairment of a JHD patient's finger tapping ability and/or preventing the slowing or the irregular performance of the Pronate-Supinate Hands test, (e) improving or maintaining, a JHD patient's gait and balance, (f) improving or maintaining, a human patient's independence in a JHD patient, (g) improving or maintaining a human patient's cognitive performance across a variety of domains a JHD patient, (h) lessening the severity of the sustained or intermittent muscle contractions associated with dystonia in a human patient in need thereof, (i) improving or maintaining motor ability in a JHD patient, (j) reducing or maintaining the level of chorea in a JHD patient, (k) improving, maintaining, or lessening the decline of a human patient's behavior and/or psychiatric state in a JHD patient, (l) reducing or maintaining a human patient's involuntary movements in a JHD patient, (m) improving or maintaining a human patient's mobility in a JHD patient, (n) improving or maintaining a JHD patient's ability to perform physical tasks, (o) improving or maintaining a JHD patient's quality of life, (p) improving, maintaining, or lessening the decline of gait and balance in a JHD patient, (q) improving, maintaining, or lessening the decline of, a human patient's independence in a JHD patient, (r) improving, maintaining, or lessening the decline of, a JHD patient's cognitive performance across a variety of domains in a human patient in need thereof, (s) reducing, maintaining, or lessening the increase of, chorea, in a JHD patient, wherein the pharmaceutical composition is to be orally administered to the patient such that a dose of 0.5 mg-300 mg of pridopidine and/or its analog or a pharmaceutically acceptable salt thereof is to be administered to the patient per day.

In an embodiment, the one or more symptoms are measured by the Clinician's Interview-based Impression of Change plus Caregiver Input (CIBIC-Plus), Physical Disability Score (PDS), Clinical Global Impression of Change (CGI-C), Unified Huntington's Disease Rating Scale (UHDRS) Independence Score (IS), HD-Quality of Life scale (HD-QoL), Multiple Sclerosis Walking Scale (MSWS-12), Physical Performance Test (PPT), hand movement score, gait and balance score, Quantitative motor (Q-Motor) assessment, timed up and go (TUG) assessment, cognitive assessment battery (CAB), symbol digit modalities test (SDMT), Stroop word reading test, Montreal cognitive assessment (MoCA) scale, Trail Making Test B assessment, or Problem Behaviors Assessment-Short form (PBA-s). In another embodiment, the one or more symptoms are measured by EQ5D-5L, Walk-12, or Modified Physical Performance Test (mPPT).

In an embodiment, the treatment of Juvenile Huntington disease in a subject comprises reducing the subject's motor impairment symptoms which are measured by the Unified Huntington's Disease Rating Scale (UHDRS) Total Motor Score (TMS).

In an embodiment, the treatment of Juvenile Huntington disease in a subject comprises reducing the subject's motor impairment symptoms which are measured by the Unified Huntington's Disease Rating Scale (UHDRS) modified Motor Score (mMS).

In an embodiment, the treatment of Juvenile Huntington disease in a subject comprises reducing the subject's motor impairment symptoms which are measured by the Unified Huntington's Disease Rating Scale (UHDRS)-Chorea score.

In an embodiment, the treatment of Juvenile Huntington disease in a subject comprises reducing the subject's motor impairment symptoms which are measured by the Unified Huntington's Disease Rating Scale (UHDRS)-Dystonia score.

As used herein, “lessening the decline of Juvenile HD” encompasses assessing the progression of HD using any of the scales and scores disclosed herein, wherein after a period of administration of pridopidine, the rate of progression of Juvenile HD as assessed by a method described herein, is decreased in a subject after a period of administration of pridopidine and/or its analog or pharmaceutically acceptable salt thereof compared with a subject who has not received the same treatment with pridopidine and/or its analog or pharmaceutically acceptable salt thereof over the same period.

In some embodiment, this invention provides a method of treating or slowing the progression of Juvenile HD in a subject, comprising administering to the subject a pharmaceutical composition comprising pridopidine and/or its analog or a pharmaceutically acceptable salt thereof.

In some embodiment, the progression of HD is defined by change in ISS from stage 1 to stage 2. In another embodiment, the progression is defined as a change in ISS from stage 2 to stage 3. In some embodiments, treating, slowing or delaying the progression of HD is measured by no change in the ISS scale. In some embodiments, treating or improving JHD is defined as a change in ISS stage from 3 to 2 or from 2 to 1.

In some embodiments, lessening the decline of the progression of Juvenile HD comprises slowing the motor deterioration in said patient compared with a subject who has not received the same treatment with pridopidine and/or its analog or pharmaceutically acceptable salt thereof over the same period. In some embodiments, lessening the decline of the progression of Juvenile HD comprises improving the motor function in said patient compared with a subject who has not received the same treatment with pridopidine and/or its analog or pharmaceutically acceptable salt thereof over the same period. In some embodiments, lessening the decline of the progression of HD comprises slowing the motor deterioration and improving the motor function in said patient compared with a subject who has not received the same treatment with pridopidine and/or its analog or pharmaceutically acceptable salt thereof over the same period.

In some embodiments, lessening the decline of the progression of Juvenile HD comprises slowing the cognitive deterioration in said patient compared with a subject who has not received the same treatment with pridopidine and/or its analog or pharmaceutically acceptable salt thereof over the same period. In some embodiments, lessening the decline of the progression of Juvenile HD comprises improving the cognitive function in said patient compared with a subject who has not received the same treatment with pridopidine and/or its analog or pharmaceutically acceptable salt thereof over the same period. In some embodiments, lessening the decline of the progression of HD comprises slowing the cognitive deterioration and improving the cognitive function in said patient compared with a subject who has not received the same treatment with pridopidine and/or its analog or pharmaceutically acceptable salt thereof over the same period.

In some embodiments, lessening the decline of the progression of Juvenile HD comprises slowing the psychiatric or behavioral deterioration in said patient compared with a subject who has not received the same treatment with pridopidine and/or its analog or pharmaceutically acceptable salt thereof over the same period. In some embodiments, lessening the decline of the progression of Juvenile HD comprises improving the psychiatric or behavioral function in said patient compared with a subject who has not received the same treatment with pridopidine and/or its analog or pharmaceutically acceptable salt thereof over the same period. In some embodiments, lessening the decline of the progression of HD comprises slowing the psychiatric or behavioral deterioration and improving the psychiatric or behavioral function in said patient compared with a subject who has not received the same treatment with pridopidine and/or its analog or pharmaceutically acceptable salt thereof over the same period.

In some embodiments, lessening the decline of the progression of Juvenile HD comprises slowing or preventing the frequency of seizures in said patient compared with a subject who has not received the same treatment with pridopidine and/or its analog or pharmaceutically acceptable salt thereof over the same period.

In some embodiments, the period of time is between 6-60 months. In some embodiments, the period of time is between 12-60 months. In some embodiments, the period of time is between 24-60 months. In some embodiments, the period of time is between 36-60 months. In some embodiments, the period of time is between 48-60 months. In some embodiments, the period of time is between 24-48 months. In some embodiments, the period of time is between 36-48 months. In some embodiments, the period of time is between 36-60 months. In some embodiments, the period of time is between 48-60 months. In some embodiments, the period of time is 6 months. In some embodiments, the period of time is 12 months. In some embodiments, the period of time is 24 months. In some embodiments, the period of time is 36 months. In some embodiments, the period of time is 48 months. In some embodiments, the period of time is 60 months. In some embodiments, the period of time is at least 6 months. In some embodiments, the period of time is at least 12 months. In some embodiments, the period of time is at least 24 months. In some embodiments, the period of time is at least 36 months. In some embodiments, the period of time is at least 48 months. In some embodiments, the period of time is at least 60 months.

In some embodiments, the administration of pridopidine results in delaying or reducing the deterioration of structural, functional, and metabolic measures in the brain. In some embodiments, these measures are evaluated by Volumetric magnetic resonance imaging (MRI) of the caudate and putamen, Functional MRI (fMRI), and Metabolic imaging using ¹⁸F-fluoro-2-deoxy-D-glucose (¹⁸FDG)-PET.

Neurofilament light proteins (NfL) may be used as a biomarker of neurodegeneration in HD patients (Byrne et al., Neurofilament light protein in blood as a potential biomarker of neurodegeneration in Huntington's disease: a retrospective cohort analysis. Lancet Neurol. 2017 August; 16(8):601-609). NfL concentrations in plasma were found to increase with advancing HD disease. Elevated NfL concentrations are associated with decreasing volume of the caudate and putamen. Thus, NfL concentrations in plasma of HD patients may provide a means for assessing and predicting neural damage in patients with HD (Byrne et al., Neurofilament light protein in blood as a potential biomarker of neurodegeneration in Huntington's disease: a retrospective cohort analysis. Lancet Neurol. 2017 August; 16(8):601-609). Additionally, results suggest that NfL in the blood could provide a reliable estimate of the concentration of NfL in the CSF (Byrne et al., Neurofilament light protein in blood as a potential biomarker of neurodegeneration in Huntington's disease: a retrospective cohort analysis. Lancet Neurol. 2017 August; 16(8):601-609).

Subjects with JHD have significantly higher plasma NfL levels than healthy controls, of approximately 7-fold. Additionally, plasma NfL levels in JHD subjects are significantly higher in JHD (approximately 1.5 hold higher than in HD gene carrier adolescents with <20 years to predicted symptom onset, false discovery rate (FDR)<0.0001) (Byrne et al., Neurofilament Light Protein as a Potential Blood Biomarker for Huntington's Disease in Children. Mov Disord. 2022 July; 37(7):1526-1531).

The invention further provides a method of maintaining, reducing, or lessening the increase of, the concentration of neurofilament light protein in a JHD patient, comprising periodically orally administering to the patient a pharmaceutical composition comprising pridopidine such that a dose of 0.5-300 mg of pridopidine is administered to the patient per day, so as to thereby maintain, decrease, or lessen the increase of, the concentration of neurofilament light protein in the human patient. In one embodiment, the dose is 0.5-5 mg bid. In another embodiment the dose is 0.5-10 mg bid. In some embodiment the dose is 1-20 mg bid. In some embodiment the dose is 5-40 mg bid. In one embodiment, the increase of the concentration of neurofilament light protein is lessened in the human patient. In another embodiment, the concentration of neurofilament light protein is maintained or decreased in the human patient.

The invention further provides a method of predicting clinical responsiveness to pridopidine therapy in a subject afflicted with JHD, the method comprising administering an amount of pridopidine and evaluating the amount of a neurofilament light protein in the subject, so as to thereby predict clinical responsiveness to pridopidine.

In one embodiment, the method further comprising predicting positive clinical responsiveness to pridopidine if the amount of the neurofilament light protein is decreased in the subject after administration of pridopidine compared to baseline. In one embodiment, the method further comprising predicting positive clinical responsiveness to pridopidine if the amount of the neurofilament light protein is maintained in the subject after administration of pridopidine relative to baseline. Baseline, in this paragraph, is the amount of the neurofilament light protein prior to administration of pridopidine.

In one embodiment, the subject is identified as a pridopidine responder if amount of the biomarker is higher than a reference value. In another embodiment, the subject is identified as a pridopidine responder if amount of the biomarker is lower than a reference value.

In some embodiments, the biomarker is mutant huntingtin (mHtt).

In an embodiment, the biomarker is the Sigma-1 receptor (SIR). In another embodiment, the biomarker is glial fibriallary acidic protein (GFAP). In an embodiment, the biomarker is tau. In another embodiment, the biomarker is phosphorylated tau. In some embodiments, the biomarker is an inflammatory marker, i.e. a pro-inflammatory cytokine, a complement factor or complement inhibitor such as clusterin, or a marker of microglial activation. In an embodiment the biomarker is interleukin 6 (IL-6). In another embodiment, the biomarker is chitinase 3-like protein 1 (CHI3L1 or YKL-40).

In some embodiments, the biomarker is a metabolic marker. In an embodiment, the biomarker is HDL-cholesterol, LDL-cholesterol, or 24(H) hydroxycholesterol.

In some embodiments, the biomarker is a neuroendocrine marker. In some embodiments, the biomarker is melatonin. In another embodiment the biomarker is cortisol.

In some embodiments, the biomarker is a marker of oxidative stress. In an embodiment, enhanced lipid peroxidation is a biomarker. In another embodiment, F2-isoprostanes are a biomarker for lipid peroxidation.

In some embodiments, the biomarker is an endogenous opioid peptide. In an embodiment, the biomarker is proenkephalin (PENK). In one embodiment, the biomarker is prodynorphin (PDYN).

In some embodiments, the biomarker is a small RNA. In some embodiments, the biomarker is a microRNA.

The invention further provides a use of an amount of pridopidine and/or its analog or a pharmaceutically acceptable salt thereof in the preparation of a medicament for treating or slowing the progression of Juvenile HD in a subject, wherein the medicament is formulated for oral administration in a daily dose of between 0.5 mg to 300 mg pridopidine and/or its analog or a pharmaceutically acceptable salt thereof.

In an embodiment, the methods disclosed herein comprise administering orally a composition comprising between 0.5 mg and 300 mg per day pridopidine and/or its analog or a pharmaceutically acceptable salt thereof. In an embodiment, the methods disclosed herein comprise administering orally a composition comprising between 5 mg and 100 mg per day pridopidine and/or its analog or a pharmaceutically acceptable salt thereof. In an embodiment, the methods disclosed herein comprise administering orally a composition comprising between 10 mg and 300 mg per day pridopidine and/or its analog or a pharmaceutically acceptable salt thereof. In another embodiment, the method comprises administering between 10 mg and 100 mg per day pridopidine and/or its analog or a pharmaceutically acceptable salt thereof. In another embodiment, between 0.5-50 mg/day, between 22.5-200 mg/day, 45-150 mg/day, 90-300 mg/day or 150-300 mg/day pridopidine, and/or its analog or a pharmaceutically acceptable salt thereof.

In an embodiment, the method comprises administering a composition comprising between a unit dose of 0.5 mg, 1 mg, 2.5 mg, 5 mg, 10 mg, 12.5 mg, 22.5 mg, 45 mg, 67.5 of pridopidine, its analog or a pharmaceutically acceptable salt thereof and is administered once a day, twice a day, three times a day, once a week, twice a week or three times a week. In another embodiment, the administration is for at least 12 weeks, at least 26 weeks, at least 52 weeks, at least 65 weeks, at least 78 weeks, at least 24 months, at least 36 months, at least 48 months, or at least 60 months. In some embodiments, the treatment is for over 60 months. In other embodiments, the treatment is for over 120 months.

In some embodiments of the compositions and methods of use thereof, a dose of 0.5 mg of pridopidine is to be administered to the patient/subject per day. In some embodiments of the compositions and methods of use thereof, a dose of 1 mg of pridopidine is to be administered to the patient/subject per day. In some embodiments of the compositions and methods of use thereof, a dose of 2.5 mg of pridopidine is to be administered to the patient/subject per day. In some embodiments of the compositions and methods of use thereof, a dose of 5 mg of pridopidine is to be administered to the patient/subject per day. In some embodiments of the compositions and methods of use thereof, a dose of 10 mg of pridopidine is to be administered to the patient/subject per day. In some embodiments of the compositions and methods of use thereof, a dose of 22.5 mg of pridopidine is to be administered to the patient/subject per day. In some embodiments of the compositions and methods of use thereof, a dose of 30 mg of pridopidine is to be administered to the patient/subject per day. In some embodiments of the compositions and methods of use thereof, a dose of 45 mg of pridopidine is to be administered to the patient/subject per day. In some embodiments of the compositions and methods of use thereof, a dose of 60 mg of pridopidine is to be administered to the patient/subject per day. In some embodiments of the compositions and methods of use thereof, a dose of 67.5 mg of pridopidine is to be administered to the patient/subject per day. In some embodiments of the compositions and methods of use thereof, a dose of 90 mg of pridopidine is to be administered to the patient/subject per day. In some embodiments of the compositions and methods of use thereof, a dose of 120 mg of pridopidine is to be administered to the patient/subject per day. In some embodiments of the compositions and methods of use thereof, a dose of 135 mg of pridopidine is to be administered to the patient/subject per day. In preferred embodiments of the methods disclosed above, the dose of 45 mg of pridopidine to be administered to the patient per day is to be administered to the patient as 22.5 mg bid. In preferred embodiments of the methods disclosed above, the dose of 90 mg of pridopidine to be administered to the patient per day is to be administered to the patient as 45 mg bid.

In some embodiments of the methods and uses for treating JHD, or in embodiments of the methods and uses for treating a subject at risk of developing JHD, the AUC0-24 achieved is equal to the AUC0-24 achieved in adult HD patients receiving 45 mg bid pridopidine. In some embodiments, AUC0-24 is 1,000 to 5,000 h*g/mL, 3,000 to 6,000 h*g/mL, 5,000 to 10,000 h*g/mL, 6,000 to 12,000 h*g/mL, or at most 15,000 h*g/mL.

In some embodiments of the methods and uses for treating JHD, or in embodiments of the methods and uses for treating a subject at risk of developing JHD, the Cmax achieved is equal to the Cmax achieved in adult HD patients receiving 45 mg bid pridopidine. In some embodiments, Cmax is 50 to 250 ng/mL, 100 to 500 ng/mL, 200 to 700 ng/mL, 300 to 800 ng/mL, or 500 to 1000 ng/mL. In some embodiments, the Cmax is at most 1200 ng/mL.

As used herein, the term “AUC” refers to the area under the plasma, serum, or blood concentration versus time curve. “AUC 0-t” refers to the area under the plasma, serum, or blood concentration versus time curve, where t (hours) is the time point of the last measurement. “AUC of infinity” refers to the area under the plasma, serum or blood concentration versus time curve extrapolated to infinity. AUC_{24,ss}, Refers to the area under the concentration-time curve from 0 to 24 hours at steady state. Units are expressed in h ng/ml.

As used herein, the term “Cmax” refers to the maximum plasma, serum or blood concentration of a drug, such as pridopidine or a pharmaceutically acceptable salt thereof, following administration of the drug.

In some embodiments, the patient is to be administered 0.5 mg pridopidine once daily (qd) for about one to two weeks and 1 mg pridopidine bid thereafter. In some embodiments, the patient is to be administered 1 mg pridopidine once daily (qd) for about one to two weeks and 2 mg pridopidine bid thereafter. In some embodiments, the patient is to be administered 2.5 mg pridopidine once daily (qd) for about one to two weeks and 5 mg pridopidine bid thereafter. In some embodiments, the patient is to be administered 5 mg pridopidine once daily (qd) for about one to two weeks and 10 mg pridopidine bid thereafter. In some embodiments, the patient is to be administered 10 mg pridopidine once daily (qd) for about one to two weeks and 10 mg pridopidine bid thereafter. In some embodiments, the patient is to be administered 25 mg pridopidine once daily (qd) for about one to two weeks and 25 mg pridopidine bid thereafter. In some embodiments, the patient is to be administered 30 mg pridopidine once daily (qd) for about one to two weeks and 30 mg pridopidine bid thereafter. In some embodiments, the patient is to be administered 45 mg pridopidine once daily (qd) for about one to two weeks and 45 mg pridopidine bid thereafter. In some embodiments of the methods disclosed above, the administration continues for at least 12 weeks, at least 26 weeks, at least 52 weeks, at least 65 weeks, or at least 78 weeks. In some embodiments of the methods disclosed above, the administration continues for 52 weeks or 78 weeks. In some embodiments of the methods disclosed above, the HD patient has been diagnosed as having at least 50 CAG repeats in the huntingtin gene. In some embodiments of the methods disclosed above the HD patient is below the age of 21 years old. In some embodiments, the HD patient is a juvenile Huntington disease (JHD) patient. In some embodiments, the juvenile HD patient is below the age of 21 years old. In some embodiments, the juvenile HD patient is below the age of 20 years old. In other embodiments, the juvenile HD patient is 18 years old or younger.

In an embodiment of the methods of the invention, the subject is below 21 years old. In an embodiment of the methods of the invention, the subject is below 18 years old. In an embodiment of the methods of the invention, the subject is between 1-21 years old, 1-20 years old, 1-19 years old, 1-18 years old, 5-18 years old or 10-18 years old. In another embodiment, the subject is human. In another embodiment the subject is a human male or human female.

In an embodiment of the methods of the invention, the subject has a UHDRS-TMS score≥25 before beginning treatment.

In an embodiment of the methods of the invention, the subject has ≥50 CAG repeats in the Huntingtin gene. In an embodiment of the methods of the invention, the subject has ≥55 CAG repeats in the Huntingtin gene. In an embodiment of the methods of the invention, the subject has ≥60 CAG repeats in the Huntingtin gene. In an embodiment of the methods of the invention, the subject has ≥50 CAG repeats in the Huntingtin gene, and the subject is below 21 years old, below 20 years old or below 18 years old.

### Pharmaceutically Acceptable Salts

The active compounds for use according to the invention may be provided in any form suitable for the intended administration. Suitable forms include pharmaceutically (i.e. physiologically) acceptable salts, and pre- or prodrug forms of the compound of the invention. Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate, the phthalate, the salicylate, the sorbate, the stearate, the succinate, the tartrate, the toluene-p-sulphonate, and the like. Such salts may be formed by procedures well known and described in the art.

### Pridopidine Analogs

In some embodiment, the composition for use in the methods of this invention comprises pridopidine or pharmaceutically acceptable salt thereof in combination with at least one of its analog compounds 1-8, or a pharmaceutically acceptable salt thereof:

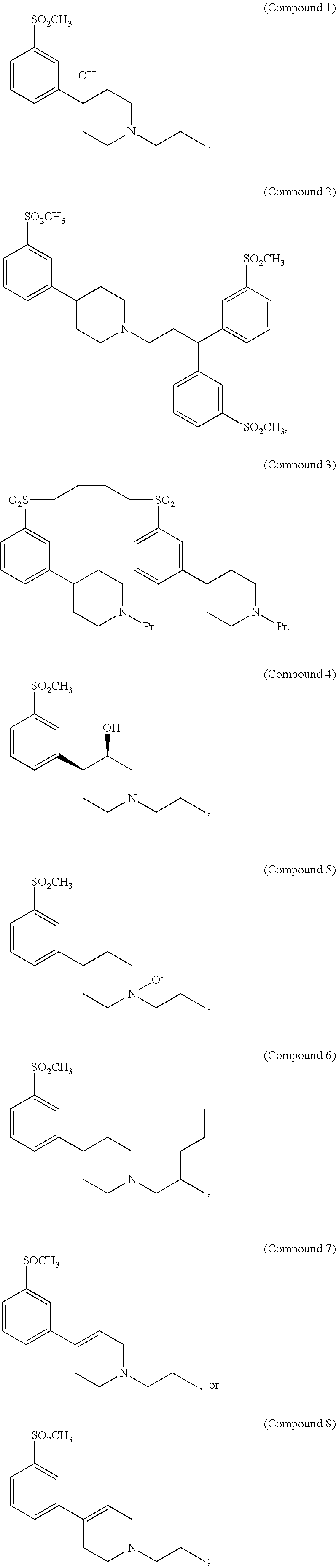

wherein the weight ratio between pridopidine or pharmaceutically acceptable salt thereof and at least one of compounds 1-8 or pharmaceutically acceptable salt thereof is in the range of 1:0.00001 to 1:0.001. In other embodiments, the weight ratio between pridopidine or pharmaceutically acceptable salt thereof and at least one of compounds 1-8 or pharmaceutically acceptable salt thereof is in the range of 1:0.0001 to 1:0.1. In other embodiments, the weight ratio between pridopidine or pharmaceutically acceptable salt thereof and at least one of compounds 1-8 or pharmaceutically acceptable salt thereof is in the range of 1:0.005 to 1:0.1. In other embodiments, the weight ratio between pridopidine or pharmaceutically acceptable salt thereof and at least one of compounds 1-8 or pharmaceutically acceptable salt thereof is in the range of 1:0.0001 to 1:0.005.

In some embodiment, the composition for use in the method of this invention comprises an amount of pridopidine or pharmaceutically acceptable salt thereof in combination with at least compound 1 or pharmaceutically acceptable salt thereof. In other embodiments, the composition for use in the method of this invention comprises an amount of pridopidine in combination with compound 1 or pharmaceutically acceptable salt thereof and compound 4 or pharmaceutically acceptable salt thereof. In other embodiments, the composition for use in the method of this invention comprises an amount of pridopidine or pharmaceutically acceptable salt thereof in combination with at least compound 2 or pharmaceutically acceptable salt thereof. In other embodiments, the composition for use in the method of this invention comprises an amount of pridopidine or pharmaceutically acceptable salt thereof in combination with at least compound 3 or pharmaceutically acceptable salt thereof. In other embodiments, the composition for use in the method of this invention comprises an amount of pridopidine or pharmaceutically acceptable salt thereof in combination with at least compound 4 or pharmaceutically acceptable salt thereof. In other embodiments, the composition for use in the method of this invention comprises an amount of pridopidine or pharmaceutically acceptable salt thereof in combination with at least compound 5 or pharmaceutically acceptable salt thereof. In other embodiments, the composition for use in the method of this invention comprises an amount of pridopidine or pharmaceutically acceptable salt thereof in combination with at least compound 6 or pharmaceutically acceptable salt thereof. In other embodiments, the composition for use in the method of this invention comprises an amount of pridopidine or pharmaceutically acceptable salt thereof in combination with at least compound 7 or pharmaceutically acceptable salt thereof. In other embodiments, the composition for use in the method of this invention comprises an amount of pridopidine or pharmaceutically acceptable salt thereof in combination with at least compound 8 or pharmaceutically acceptable salt thereof.

In other embodiments, the analog compounds 1-8 of pridopidine and their methods of preparation may be found in U.S. Pat. Nos. 10,130,621 and 10,406,145 the entire content of each of which is hereby incorporated by reference.

In an embodiment, the pharmaceutically acceptable salt of pridopidine comprises the hydrochloride, hydrobromide, nitrate, perchlorate, phosphate, sulphate, formate, acetate, aconate, ascorbate, benzenesulphonate, benzoate, cinnamate, citrate, embonate, enantate, flumarate, glutamate, glycolate, lactate, maleate, malonate, mandelate, methanesulphonate, naphthalene-2-sulphonate, phthalate, salicylate, sorbate, stearate, succinate, tartrate or toluene-p-sulphonate salt.

In an embodiment, the pharmaceutically acceptable salt of compounds 1-8 comprises the hydrochloride, hydrobromide, nitrate, perchlorate, phosphate, sulphate, formate, acetate, aconate, ascorbate, benzenesulphonate, benzoate, cinnamate, citrate, embonate, enantate, fumarate, glutamate, glycolate, lactate, maleate, malonate, mandelate, methanesulphonate, naphthalene-2-sulphonate, phthalate, salicylate, sorbate, stearate, succinate, tartrate or toluene-p-sulphonate salt.

### Pharmaceutical Compositions

While the compounds for use according to the invention may be administered in the form of the raw compound, it is preferred to introduce the active ingredients, optionally in the form of physiologically acceptable salts, in a pharmaceutical composition together with one or more adjuvants, excipients, carriers, buffers, diluents, and/or other customary pharmaceutical auxiliaries.

In an embodiment, the invention provides pharmaceutical compositions comprising the active compound, and/or its analog or pharmaceutically acceptable salt or derivative thereof, together with one or more pharmaceutically acceptable carriers therefore, and, optionally, other therapeutic and/or prophylactic ingredients know and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in dragé, in multiparticulate, in beads, in granules, in powder, or in liquid form, and parenteral administration, in particular cutaneous, subcutaneous, intramuscular, or intravenous injection. The pharmaceutical composition of the invention can be manufactured by the skilled person by use of standard methods and conventional techniques appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, PA).

As used herein, “effective” as in an amount effective to achieve an end means the quantity of a component that is sufficient to yield an indicated therapeutic response without undue adverse side effects (such as toxicity, irritation, or allergic response) commensurate with a reasonable benefit/risk ratio when used in the manner of this disclosure. For example, an amount effective to treat a movement disorder. The specific effective amount varies with such factors as the particular condition being treated, the physical condition of the patient, the type of mammal being treated, the duration of the treatment, the nature of concurrent therapy (if any), and the specific formulations employed and the structure of the compounds or its derivatives.

As used herein, an amount of pridopidine as measured in milligrams refers to the milligrams of pridopidine (4-[3-(methylsulfonyl) phenyl]-1-propyl-piperidine) present in a preparation, regardless of the form of the preparation. For example, a unit dose containing “90 mg pridopidine” means the amount of pridopidine in a preparation is 90 mg, regardless of the form of the preparation. Thus, when in the form of a salt, e.g., pridopidine hydrochloride, the weight of the salt form necessary to provide a dose of 90 mg pridopidine would be greater than 90 mg due to the presence of the salt.

As used herein to “treat” or “treating” encompasses, e.g., reducing a symptom, inducing inhibition, regression, or stasis of the disorder and/or disease. As used herein, “inhibition” of disease progression or disease complication in a subject means preventing or reducing the disease progression and/or disease complication in the subject.

This invention will be better understood by reference to the Experimental Details which follow, but those skilled in the art will readily appreciate that the specific experiments detailed are only illustrative of the invention as described more fully in the claims which follow thereafter.

### EXAMPLES

### Example 1: A Clinical Study Evaluating the Pharmacokinetics, Safety, and Efficacy of Pridopidine in Juvenile HD Patients

A double-blind, placebo-controlled trial to evaluate the pharmacokinetics, safety, and efficacy of pridopidine is performed in children and adolescents aged 2-17. Inclusion criteria includes UHDRS TMS>10. A total of 24-30 male and female adolescent and childhood-onset JHD patients (2 years and older) are planned to be enrolled—up to 24 adolescents (aged 10-17) and at least 6 childhood JHD patients (aged 2-10).

The study is conducted in two sequential parts. Part 1 (4 weeks) is conducted to establish the dose to be used in Part 2 (52 weeks).

A single dose level of pridopidine is administered orally twice daily. A dose level which is expected to yield, in pediatric JHD patients, an exposure of Cₘₐₓ between 200-800 ng/ml and/or AUC₀₋₂₄ of 1000-10,000 h×ng/ml.

Part 1 comprises 6 patients treated for a total of 4 weeks (based on dose estimated in the PopPK model) (2 weeks up titration once daily (qd) followed by 2 weeks of twice-daily (bid) (full daily dose). Once all 6 patients have completed their participation in Part 1 (and reached steady state on the full dose), and data from their 4-week PK profile have been collected, PK analysis is performed to confirm the appropriate dose to be used in Part 2.

In Part 2 of the study, subjects are enrolled after PK analysis of Part 1 is completed. Part 2 patients are treated at the dose level confirmed after the Part 1 PK analysis.

The safety and tolerability of pridopidine are assessed throughout the study via ongoing collection of adverse events, and evaluation of Safety laboratory assessments, Columbia Suicide Severity Rating Scale (C-SSRS) and ECG monitoring at specific visits.

### Primary Endpoint:

To evaluate the effect of pridopidine on motor function using Unified Huntington Disease Rating Scale (UHDRS)-Total Motor Score (TMS). UHDRS-TMS is evaluated at baseline (Day 1) and at Weeks 4, 13, 26 and 52.

For all subjects, PK analyses are performed at baseline, and at each study visit (2 hours post-dose).

### Secondary Endpoints:

To evaluate the effect of pridopidine on the disease biomarker plasma neurofilament light (NfL) protein. NfL is evaluated at baseline and at Weeks 13, 26 and 52.

To evaluate the effect of pridopidine on imaging parameters using Volumetric magnetic resonance imaging (MRI) of the caudate and putamen, Functional MRI (fMRI), and Metabolic imaging using 18F-fluoro-2-deoxy-D-glucose (18FDG)-PET. Imaging is performed at baseline and at Weeks 26 and 52.

To evaluate the effect of pridopidine on motor function using Quantitative motor (Q-Motor) finger tapping (Digitomotography).

To evaluate the effect of pridopidine on cognition using the Leiter International Performance Scale and the Peabody Picture Vocabulary Test.

Seizure frequency and duration.

Pediatric Quality of Life Inventory (PedsQL).

To evaluate the effect of pridopidine on psychiatric measures using Problem Behaviors Assessment-Short Form (PBA-s).

To assess the safety and tolerability of pridopidine over a 52-week treatment period via ongoing collection of adverse events (AEs) and Safety and tolerability of pridopidine are assessed throughout the study via ongoing collection of adverse events and Columbia-Suicide Severity Rating Scale (C-SSRS), which is assessed at every in-clinic visit. In addition, evaluation of safety laboratory parameters, and electrocardiogram (ECG) monitoring are performed on Day 1 and in Weeks 4, 13, 26 and 52.

To assess the PK of pridopidine and its main metabolite over a 52-week treatment period. For all patients, PK analyses are performed at baseline and at each study visit (2 hours post-dose). A full PK profile is performed for the first 6 patients in Part 1 of the study at baseline (Day 1), at Week 2 (steady state on once daily) and at Week 4 (steady state on full, twice-daily dose). PK samples are collected at the following timepoints: t=0 (pre-dose), 0.5, 1, 2, 4, 8, 10, 12 and 24-hours post-dose.

### Example 2: Case Study: Pridopidine Treatment in an HD Patient with CAG≥50

This case study reports on the effects of pridopidine 45 mg bid in a patient with CAG≥50. CAG≥50 is associated with juvenile HD. The effects of pridopidine were compared to the effects of placebo on patients with CAG≥50 (n=4).

In the following Methods and Results sections 45 mg bid pridopidine refers to a composition comprising pridopidine HCl with Compound 1 (in the range of 0.07% w/w-0.09% w/w from pridopidine) and Compound 4 (in the range of 0.10-0.12% w/w from pridopidine). 45 mg refers to the base form of pridopidine.

### Methods:

All patients began treatment with a 2-week titration period (45 mg once a day) followed by full-dose treatment for 63-78 weeks.

Efficacy was evaluated using the following clinical endpoints: Change from Baseline to Week 78 in composite UHDRS (cUHDRS) total score, Total Functional Capacity (UHDRS-TFC) score and Stroop Word Reading (SWR) testing for cognitive assessment.

### Results:

### UHDRS-TFC Score:

The UHDRS-TFC score is a commonly used and regulatory-accepted clinical scale used for assessing the functional capacity of subjects with HD and monitoring the disease's progression and severity over time. This score ranges from 0 to 13 and higher scores indicate better functional capacity. A score of 13 means that the individual is entirely functional with no indications of impairment, while a score of 0 indicates complete dependence on others for daily activities.

All participants in this study had CAG repeats greater than or equal to 50. Participants receiving placebo demonstrated a decline in TFC at all timepoints evaluated. The HD participant receiving pridopidine 45 mg bid (n=1) showed a marked improvement in TFC compared to HD participants (n=4) in the placebo group, up to 78 weeks (Table 1). Pridopidine with compounds 1 and 4 showed a positive treatment effect compared to placebo with change vs placebo of 2.99 points at 26 weeks, 3.06 points at 39 weeks, 3.00 points at 52 weeks, 2.90 points at 65 weeks and at 78 weeks, 3.45 points change (Positive change indicates improvement) (Table 1).

TABLE 1
Pridopidine improves TFC in Juvenile HD patients with CAG repeat
length >=50 compared to placebo up to 78 weeks

| | Placebo | | Pridopidine 45 mg bid | | Pridopidine 45 mg |
|---|---|---|---|---|---|
| | | LS Mean | | LS Mean | bid vs Placebo |
| | | change from | | change from | LS Mean change vs |
| Week | N | baseline | N | baseline | placebo |
| 26 | 4 | −0.027 | 1 | 2.96 | 2.99 |
| 39 | 4 | −0.097 | 1 | 2.96 | 3.06 |
| 52 | 4 | −0.087 | 1 | 2.91 | 3.00 |
| 65 | 4 | −0.289 | 1 | 2.61 | 2.90 |
| 78 | 4 | −0.512 | 1 | 2.93 | 3.45 |

Data from a study case. Effect of pridopidine 45 mg bid in combination with compound 1 and 4 and placebo in HD patients with CAG repeat length >=50, without antidopaminergic drugs. Positive change in TFC indicates improvement.

cUHDRS:

The composite Unified Huntington's Disease Rating Scale (cUHDRS) is a clinical scale used for evaluating the severity and progression of Huntington's disease (HD).

The cUHDRS is a composite scale assessing overall progression of independent key aspects of HD: functional ability, motor symptoms, and cognitive function. cUHDRS comprises four subscales: Total Functional Capacity (TFC), Total Motor Score (TMS), and 2 cognitive measures: the Symbol-Digit Modality Test (SDMT), and Stroop Word Reading (SWR) Test.

The cUHDRS shows outstanding test-retest reliability (ICC=0.96) and is superior to its individual components in terms of sensitivity to disease stage and longitudinally providing greater statistical power to detect clinical benefit (Schobel, Scott A., et al. “Motor, cognitive, and functional declines contribute to a single progressive factor in early HD.” Neurology 89.24 (2017): 2495-2502).

The cUHDRS score is a validated scale used for tracking disease progression and evaluating treatment effectiveness in subjects with HD. Higher scores on cUHDRS indicates better overall disease state. By measuring multiple domains, the cUHDRS provides a global and comprehensive assessment of the disease stage.

All participants in this study had CAG repeats greater than or equal to 50. Participants on placebo showed the predictable decline in cUHDRS at every timepoint up to 78 weeks. Pridopidine with compounds 1 and 4 (n=1) showed positive treatment effect compared to placebo (n=4) on cUHDRS up to 78 weeks. Pridopidine with compounds 1 and 4 showed positive treatment effect compared to placebo with change vs placebo of 2.52 points at 26 weeks, 2.66 points at 39 weeks, 2.49 points at 52 weeks, 2.32 points at 65 weeks and at 78 weeks, 2.52 points change (Positive change indicates improvement) (Table 2).

TABLE 2
Pridopidine improves overall disease progression in
Juvenile HD patients with CAG repeat length >=50
compared to placebo up to 78 weeks as measured by cUHDRS

| | Placebo | | Pridopidine 45 mg bid | | Pridopidine 45 mg |
|---|---|---|---|---|---|
| | | LS Mean | | LS Mean | bid vs Placebo |
| | | change from | | change from | LS Mean change vs |
| Week | N | baseline | N | baseline | placebo |
| 26 | 4 | −0.72 | 1 | 1.79 | 2.52 |
| 39 | 4 | −0.82 | 1 | 1.83 | 2.66 |
| 52 | 4 | −0.82 | 1 | 1.67 | 2.49 |
| 65 | 4 | −0.91 | 1 | 1.41 | 2.32 |
| 78 | 4 | −1.09 | 1 | 1.42 | 2.52 |

Data from a study case. Effect of pridopidine 45 mg bid in combination with compound 1 and 4 and placebo in HD patients with CAG repeat length >=50, without antidopaminergic drugs CAG ≥50. Positive change indicates improvement.

### SWR Score:

The Stroop Word Reading (SWR) test is a validated and widely used clinical measure of cognitive function. The SWR assesses the ability to inhibit cognitive interference and is commonly used to evaluate executive function, including cognitive flexibility and attentional control.

During the SWR test, individuals were asked to read a list of color names that are printed in colored ink, where the ink color does not match the word. This creates a cognitive conflict that requires the inhibition of a prepotent response to read the word, making the test challenging.

The SWR score is determined by assessing the time it takes to read the list of words and the number of errors made. Higher scores on the SWR test indicate better cognitive performance.

HD participants with CAG repeat length≥50 were treated with pridopidine 45 mg bid (n=1) or placebo (n=4) for 78 weeks. Participants received placebo showed the predictable, marked decline in cognitive function as assessed by the SWR. However, patients treated with pridopidine with compounds 1 and 4 showed positive treatment effect compared to placebo at every timepoint, up to 78 weeks. Pridopidine with compounds 1 and 4 showed positive treatment effect compared to placebo with change vs placebo of 14.58 points at 26 weeks, 15.38 points at 39 weeks, 15.44 points at 52 weeks, 12.35 points at 65 weeks and at 78 weeks, 14.28 points change (Positive change indicates improvement) (Table 3).

TABLE 3
Pridopidine improves cognitive function in Juvenile HD patients with
CAG repeat length >=50 compared to placebo up to 78 weeks

| | | | | | Pridopidine |
|---|---|---|---|---|---|
| | | | Pridopidine | | 45 mg bid with |
| | | | 45 mg bid with | | compounds |
| | Placebo | | compounds 1 and 4 | | 1 and 4 vs |
| | | LS Mean | | LS Mean | Placebo |
| | | change from | | change from | LS Mean change |
| Week | N | baseline | N | baseline | vs placebo |
| 26 | 4 | −5.79 | 1 | 8.78 | 14.58 |
| 39 | 4 | −6.35 | 1 | 9.03 | 15.38 |
| 52 | 4 | −5.97 | 1 | 9.47 | 15.44 |
| 65 | 4 | −5.51 | 1 | 6.84 | 12.35 |
| 78 | 4 | −6.74 | 1 | 7.53 | 14.28 |

Data from case study. Effect of pridopidine 45 mg bid in combination with compound 1 and 4 and placebo in HD patients with CAG repeat length >=50, without antidopaminergic drugs CAG ≥50. Positive change indicates. improvement.

### SUMMARY

In Juvenil HD patients, who have CAG repeat length>=50, pridopidine 45 mg bid demonstrates a marked improvement. in functional capacity, cognitive function, and disease progression up to 78 weeks.

1. A method of treating or lessening the progression of Juvenile Huntington disease in a subject, or to maintaining or reducing one or more symptoms or signs of Juvenile Huntington disease (JHD) in a subject; comprising orally administering to the subject a pharmaceutical composition comprising pridopidine or a pharmaceutically acceptable salt thereof.

2. The method of claim 1 wherein the one or more symptoms or signs are selected from the group consisting of motor function impairment, cognitive impairment, a physical symptom, a mental symptom, an emotional symptom, a behavioral symptom, a psychiatric symptom, depression, anxiety, aggression, impulsivity, seizures, and reduced lifespan.

3. The method of claim 2 wherein the symptoms or signs of Juvenile HD comprise motor function.

4. The method of claim 2 wherein the symptoms or signs of Juvenile HD comprise cognitive function in a Juvenile Huntington disease subject.

5. The method of claim 2 wherein the symptoms or signs of Juvenile HD comprise behavioral function in a Juvenile Huntington disease subject.

6. The method of claim 2 wherein the symptoms or signs of Juvenile HD comprise psychiatric function in a Juvenile Huntington disease subject.

7. The method of claim 1 wherein the composition further comprises at least one of pridopidine's analog of compounds 1-8, or pharmaceutically acceptable salt thereof:

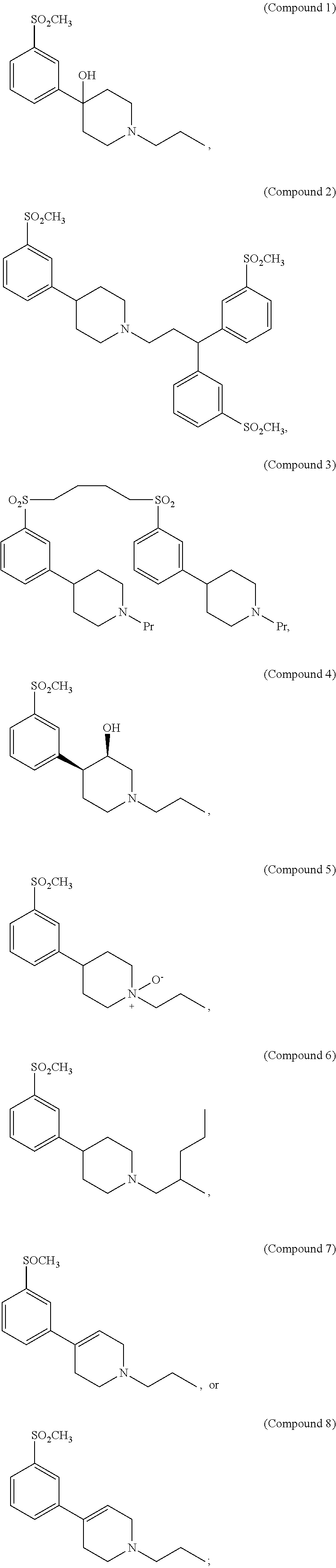

8. The method of claim 1, wherein the human patient experiences onset of symptoms before the age of 20.

9. The method of claim 1, wherein the human patient has ≥50 CAG repeats in the Huntingtin gene.

10. The method of claim 1, wherein the pharmaceutical composition is administered once a day, twice per day, or three times per day.

11. The method of claim 2 wherein impairment of the human patient's motor function is measured by the Unified Huntington's Disease Rating Scale (UHDRS) Total Motor Score (TMS).

12. The method of claim 2 wherein impairment of the human patient's motor function is assessed by the Quantitative Motor (Q-Motor) battery of tests.

13. The method of claim 1 wherein the method further comprises administering a pharmaceutical composition comprising an anti-seizure drug, an anti-depression drug, an anxiolytic, tetrabenazine, deutetrabenazine, or a mutant Huntingtin (mHtt) lowering agent.
